(19) United States
(12) Patent Application Publication
(10) Pub. No.: US 2014/0018654 A1
(43) Pub. Date: Jan. 16, 2014

(54) Color change wound dressing

(71) Applicant: Thomas J. Drury, Tolland, CT (US)

(72) Inventor: Thomas J. Drury, Tolland, CT (US)

(73) Assignee: Tolland Development Company, LLC

(21) Appl. No.: 13/507,581

(22) Filed: Jul. 12, 2012

(51) Int. Cl. A61B 5/1477; B32B 38/14

(52) U.S. Cl. 600/362; 156/256

(57) ABSTRACT

The present invention is directed toward a wound dressing comprising a foam body made of polyvinyl acetal material treated with a plurality of colored biocidal dyes with at least one of the dyes being gram positive and at least one of the dyes being gram negative to dye the body a distinct color. The body has at least one planar surface and ranges from about 1 mm to about 3 mm in thickness and has an outer adhesive section secured to the body. The body when placed in contact with an infected wound site changing color.

### RELATED APPLICATIONS

There are no related applications.

### STATEMENT REGARDING FEDERALLY SPONSORED RESEARCH OR DEVELOPMENT

None

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention relates generally to a wound care dressing and more specifically relates to a wound care dressing constructed of a synthetic polymer foam body preferably of polyvinyl acetal (PVA) having a combination biocidal dye with non-oxidant gram positive and gram negative characteristics, preferably Methelyne Blue and Gentian Violet biocidal dyes bound to the foam, the foam body being secured to an outer adhesive plastic member at the outer edges of the foam body. The foam body ranges from about 1 mm to about 3 mm in thickness and when placed on an open wound, color changes to indicate infection at the wound site.

2. Background of the Invention

There are a number of major problems encountered in present day wound dressings. Such wound dressings are primarily of the cellulose type and the sponge type. Cellulose dressings have probably been used in some form since the beginning of recorded history and are basically constructed of cellulose materials such as rayon and cotton. These wound dressings leave linting fibers, allow bacterial growth adjacent the wound and allow wound growth extension into the fibers of the dressing. Removal of the dressing also causes tearing of the wound when the dressing is removed and can leave fibrous fragments in the wound. Because of the fiber spacing in such dressings they also do not present a barrier against direct exposure to air and organisms carried in the air. These wound dressings do not provide a color change allowing a nurse or technician to observe infection at the wound site.

Advances in the development of synthetic polymers have produced numerous changes in wound care dressings resulting in polymeric foams, polymeric films, particulate and fibrous polymers, hydrogels and hydrocolloids. These developments have resulted in sponge type dressings being widely used but unfortunately these dressings also suffer from wound growth into the cells of the sponges, failure to kill bacteria and other infectious agents, lack of absorption and wound tearing problems which occur when the dressings are removed.

A number of sponge type dressings and medical devices have been constructed of PVA material. These products have a significant percent of their alcohol functions acetalized and are open celled, highly water absorbent porous flexible material that wick aqueous solutions quickly.

U.S. Pat. No. 4,098,728 issued Jul. 4, 1978 discloses the use of polyvinyl acetal material having a fast wicking and high liquid holding capacity for medical usage.

U.S. Pat. No. 5,071,648, issued on Dec. 10, 1991 discloses a polyvinyl acetal material with a complex of iodine which forms a sponge releasing controlled amounts of iodine sufficient to kill germ cells with minimum toxicity to the surrounding tissue. Likewise, U.S. Pat. No. 5,744,150 issued on Apr. 28, 1998 and U.S. Pat. No. 5,928,665 issued Jul. 27, 1999 disclose a method for producing an antimicrobial iodine polyvinyl acetal sponge which is soaked in an aqueous bath of 20% to 70% triethylene glycol. The resultant product is a wound dressing including an iodine complexed polyvinyl acetal sponge material in which alkylene glycol is applied to the surface of the sponge to soften the sponge and impart a yellow-gold coloration onto the outer surface of the sponge indicating the activation of the antimicrobial elements complexed in the sponge material. U.S. Pat. No. 5,810,755 issued Sep. 22, 1998 discloses a medicated wound dressing with an open cell foam polymeric compound of polyvinyl alcohol complexed with elemental iodine. None of these references exhibit a color change of the wound dressing indicating infection at the wound site.

U.S. Pat. No. 5,554,659 issued Sep. 10, 1996 and U.S. Pat. No. 5,556,391 issued Sep. 17, 1996 are directed toward a molded porous polyvinyl alcohol sponge including an outer skin having an average pore size smaller than the interior portion of the product capable of absorbing and passing water to the interior portions of the sponge.

U.S. Pat. No. 5,811,471, issued Sep. 22, 1998 discloses a polyvinyl acetal polymer sponge which has a germicidal disinfectant dye bound thereto which is used as a tampon while U.S. Pat. No. 5,447,505 issued Sep. 5, 1995 discloses the use of polyvinyl acetal sponge surgical dressing. U.S. Pat. No. 6,183,764 issued Feb. 6, 2011 and U.S. Pat. No. 6,361,786 issued Mar. 26, 2002 are also directed toward a polyvinyl acetal polymer material which have a plurality of organic dyes absorbed therein to provide microbiocidal properties. Polyvinyl acetal is already used in the prior art for nasal packings and other surgical packings. Another wound sponge dressing shown in U.S. Pat. No. 5,466,231 issued Nov. 14, 1995 adheres or laminates a layer or sheet of perforated polyethylene to the lateral surfaces of a polyvinyl acetal sponge to allow moisture transfer.

U.S. Pat. No. 6,613,347 issued Sep. 2, 2003 is directed toward a polyvinyl acetal sponge with a smooth outer low durometer silicone skin having less porosity then the foam center. The PVA sponge is washed free of formaldehyde, dried and hydrated and a thin coating of less than 1 mm low durometer silicone is applied to the surface of the sponge and heated at a low temperature ranging from 100.degree. F. and 150.degree. F. over 8 to 16 hours to cure the silicone skin bonding it to the sponge increasing the tear strength of the skin while preserving elasticity. The composite wound dressing allows moisture adsorption through the skin into the PVA sponge body but presents an outer surface precluding wound growth into the sponge material.

None of the aforementioned references exhibit color change when in contact with the wound to indicate if the site is infected. The present inventive wound dressing changes its base color to white as an indicator that the wound site is infected.

The present invention solves the above noted problems with wound care sponges in a manner not disclosed in the known prior art and serves as an indication of infection by undergoing color changes.

### SUMMARY OF THE INVENTION

The present invention is directed toward a polyvinyl acetal (PVA) wound care sponge dressing having a thickness ranging from about 1 mm to 3 mm and an adhesive skin secured to the edge of the dressing body. The PVA sponge is washed free of formaldehyde, and a gram negative biocidal Methelyne Blue dye and a gram positive Gentian Violet dye are bonded to the PVA material. The sponge is dried and hydrated and an outer strip of adhesive skin is secured to the outer edge surface of the sponge and heated at a low temperature over 8 to 16 hours bonding it to the sponge while maintaining its adhesive qualities and strength. A removable plastic strip covering is then placed over the outer strip. The composite wound dressing which does not require hydration and which is applied dry, allows moisture adsorption into the PVA sponge body causing the dressing to expand indicating wound leakage and turns white indicating infection at the wound site.

It is an object of the invention to provide a dry wound care dressing allowing for the passage of moisture from the wound into the sponge body.

It is another object of the invention to provide a wound care dressing having an outer adhesive surface of different material from the body of the dressing allowing the dressing to be applied to a patient's body.

It is yet another object of the invention to provide a wound care dressing which softens as it comes into contact with normal tissue moisture.

It is still another object of the invention to provide a wound dressing which allows comfortable easy application of the dressing to the wound and upon removal leaves the wound area clean with minimal tearing of the wound area.

It is another object of the invention to provide a wound dressing which applies a biocidal material covering a full bacteria spectrum at a wound site.

It is yet another object of the invention to provide a wound dressing which exhibits a color change when the wound site is infected.

In the accompanying drawings, there is shown an illustrative embodiment of the invention from which these and other objectives, novel features and advantages will be readily apparent.

### BRIEF DESCRIPTION OF THE DRAWINGS

FIG. 1 is a plan view of the inventive wound care dressing;

FIG. 2 is an enlarged cross sectional view of an inventive wound care dressing shown in FIG. 1 taken along lines 2′-2′; and

FIG. 3 is a perspective view of the inventive wound care dressing placed on a patient showing color change.

These and other objects, advantages, and novel features of the present invention will become apparent when considered with the teachings contained in the detailed disclosure along with the accompanying drawings.

### DESCRIPTION OF THE INVENTION

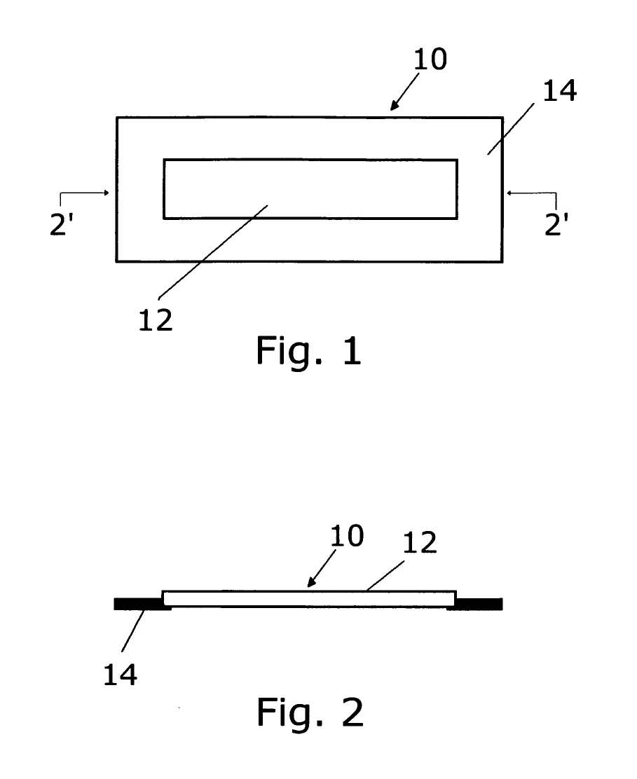

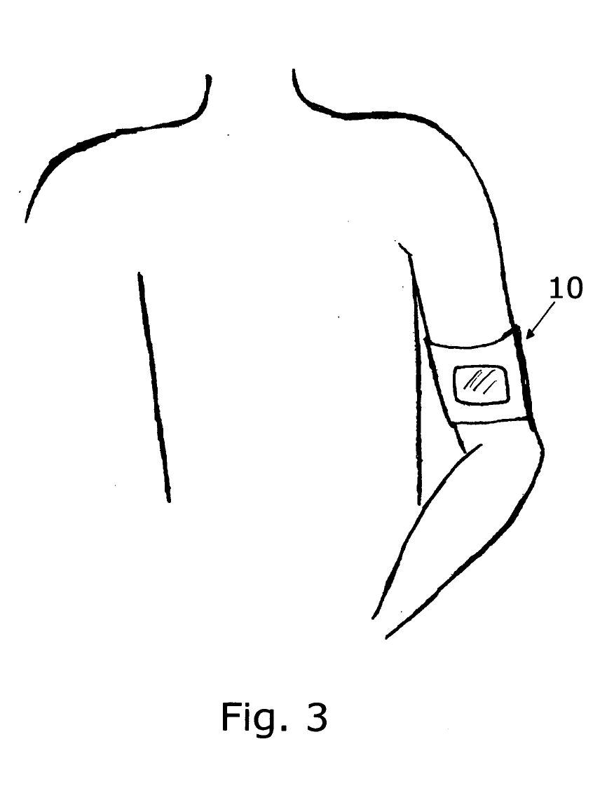

The best mode and the preferred embodiment of the novel wound care dressing is shown generally in FIGS. 1 through 3.

FIG. 1 illustrates a wound care dressing or sponge 10 formed with an inner porous polyvinyl acetal sponge or foam material body 12 with a substantially planar top and bottom surface having a thickness ranging from about 1 mm to about 3 mm in thickness, preferably about 2 mm in thickness. It is also envisioned that polyurethane foam, polyurethane ester foam, polyurethane ether foam and polyethylene foams could be used providing the same are properly cleaned to remove the foaming agent and open up dye receptor binding sites. An outer adhesive section 14 is secured to a flat surface of the sponge body. In the preferred embodiment, the polyvinyl acetal material of the body 12 is about 99.5% by weight. A gram positive Methelyne Blue dye of about 0.025% by gram weight is bonded to the sponge material and a gram negative Gentian Violet dye of about 0.025% by gram weight is bonded to the sponge material.

Alternatively, other bactericidal dyes such as gram negative Dimethyl Methelyne Blue (blue color); gram negative New Methelyne Blue (blue color); gram positive Brilliant Green (green color); gram positive Malachite (green color); gram positive Acriflavine (orange color) and gram positive Quinacrine (yellow color) may be added in the same gram weight noted above, namely 0.025% as long as one dye is gram positive and the other dye is gram negative so that the full spectrum of bacteria and other microbial entities can be acted upon. It has been found that when the aforementioned dyes are added to the foam at a concentration of at least 0.025% gram weight that this concentration provides a complete biocidal effect throughout the bacterial spectrum and the combination of gram positive and gram negative dyes respectively act on the full spectrum of bacteria. No oxidizing dyes or oxidizing materials such as silver oxide should be added to the foam material as the shelf life and biocidal effectiveness of the foam material will be compromised.

The body 12 forming the center section of the wound dressing and outer adhesive section or strip 14 of the dressing is then heated to between 100° F. and 150° F. for between 8 to 16 hours which cures the outer adhesive section to the sponge body 12. The outer adhesive section 14 around the dressing can be made of medical grade adhesive preferably urethane, polypropylene or silicone. The body or center section 12 ranges from 1 mm to 3 mm in thickness, preferably 2 mm. When silicone is used for the outer adhesive section 14, the silicone is commercially available from GE Silicones under the product designation LIM6010.

The GE silicone product has a specific gravity of 1.05, a viscosity (cps) of about 30,000 and a Shore A Durometer ranging from 10 when molded at 30 seconds to 15 at one hour at 350° F. It is believed that the durometer increases when baked at a lower temperatures for an increased duration and that this curing causes both chemical and mechanical bonding between the PVA material and the silicone. If desired, the sponge and silicone can be heated and quickly cured from 1 to 30 seconds at 300° F. and 450° F. to provide a skin for the outside strip with greatly reduced or no porosity. Polyvinyl acetal has been selected because of its absorbability of fluids, the ability to be treated with microbial materials and because it can absorb shock through the flexible cell structure of the material while retaining rigidity allowing it to maintain shape when placed over a wound. The wound care sponge 10 center section of polyvinyl acetal wicks fluid from the body wound while the outer section 14 prevents tissue growth and allows ease of removal of the wound dressing from the patient.

The base polyvinyl acetal material is heated and solubilized at 190 degrees Fahrenheit, mixed with a cross linking agent and catalyzed and placed on a sheet. After removing the sheet of PVA material it is washed with a deionized-water carrier several times to remove the foaming formaldehyde so that the formaldehyde is undetectable (under 1 part per million) by high pressure liquid chromatography. The PVA material is dried and hydrated and then rung out to remove any excess moisture. The PVA sponge body used is a white open-celled sponge, with instantaneous fluid wicking, an absorptive capacity of up to 27 times its weight in fluids and a retained fluid capacity of up to 16 times its own weight in fluids. Methelyne Blue is placed in solution and the sheet of sponge material is placed in the solution bath with Methelyne Blue bonding to the sponge material. The resultant dyed sponge material is dried. Gentian Violet is placed in solution and the sheet of sponge material is placed in the solution bath with the Gentian Violet bonding to the sponge material. The final dried sponge or foam material is 99.5% polyvinyl sponge by weight 0.025% Methelyne Blue by weight and 0.025% Gentian Violet by weight and has a bright blue color. The formed sheet of

PVA material is preferably about 2 mm but within the range of 1 mm to 3 mm in thickness and the sheet can be cut in a pad length of 6 to 10 inches, with a preferred length of 8 inches and a width of about 2.5 inches to 3 inches preferably 2.75 inches.

Any of a variety of substances can be later introduced into the PVA after washing by soaking or immersing the PVA in a solution of the desired substance(s) followed by drying of the PVA. Substances which can be readily incorporated in the PVA by this or any other suitable manner include antimicrobials and/or antibiotics such as erythromycin, bacitracin, neomycin, penicillin, polymyxin B, tetracycline viomycin, chloromycetin and streptomycins, cefazolin, ampicillin, tobramycin, clindamycine and gentamycin, etc.; amino acids, peptides, vitamins, co-factors for protein synthesis; hormones; synthesizers; enzymes such as collagenase, peptidases, oxidases, etc.; angiogenic drugs and polymeric carriers containing such drugs; biocompatible surface active agents; antigenic agents. The dressing is applied dry and will soften as it comes into contact with normal tissue moisture. The color of the body 12 of the wound dressing is blue and infection at the wound site turns the dressing white.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

1. A wound dressing comprising a body made of polyvinyl acetal foam material treated with a plurality of colored biocidal non-oxidating dyes, each dye having an individual gram positive characteristics or a gram negative characteristics, said wound dressing body having a composition by weight of about 99.5% polyvinyl acetal material, about 0.025% by weight at least one gram positive dye and about 0.025% by weight at least one gram negative dye, said dyes imparting in the wound dressing body a distinct color and binding to the polyvinyl acetal material, said body ranging from about 1 mm to about 3 mm in thickness and an outer adhesive section secured to said body, said body when placed in contact with an infected wound site changing color.

2. (canceled)

3. A wound dressing as claimed in claim 1 wherein said foam body is originally colored blue after treatment with said colored biocidal dyes and has a color change when placed on an infectious wound site to white.

4. (canceled)

3. A wound dressing as claimed in claim 1 wherein said dyes are a gram negative Methelyne Blue dye and a gram positive Gentian Violet dye.

4. A wound dressing as claimed in claim 1 wherein said dyes are taken from a group of gram negative dyes consisting of Dimethyl Methylene Blue and New Methylene Blue and a group of gram positive dyes consisting of Brilliant Green dye, Malachite Green dye, Acriflavine dye and Quinacrine dye.

7. (canceled)

5. A wound dressing as claimed in claim 1 wherein said outer adhesive section is silicone.

6. A wound dressing as claimed in claim 1 wherein said outer adhesive section is taken from a group consisting of urethane, polypropylene and silicone.

10. (canceled)

7. A wound dressing as claimed in claim 1 wherein said polyvinyl acetal wound dressing is about 2 mm in thickness.

12. (canceled)

8. A wound dressing comprising a body section made of foamed polyvinyl acetal foam material treated with a plurality of colored non-oxidating biocidal dyes with at least one of said dyes being gram positive and at least one of the said dyes being gram negative, said dyes coloring the body section a distinct color, said body section ranging from about 1 mm to about 3 mm in thickness and an outer adhesive section secured to said body section providing means to secure said wound dressing to a patient, said body section when placed in contact with an infected wound site changing color indicating the infection.

9. A wound dressing as claimed in claim 8 wherein said wound dressing body section has a composition by weight of about 99.5% polyvinyl acetal material, about 0.025% by weight Methylene Blue dye and about 0.025% by weight Gentian Violet dye.

10. A wound dressing as claimed in claim 8 wherein said wound dressing body section has a composition by weight of about 99.5% polyvinyl acetal material, about 0.025% by weight biocidal dye taken from a group consisting of gram negative dyes Methelyne Blue, Dimethyl Methylene Blue and New Methylene Blue and about 0.25% by weight of a biocidal dye taken from a group consisting of gram positive dyes Brilliant Green dye and Malachite Green dye, and Acriflavine dye and Quinacrine dye.

16. (canceled)

11. A wound dressing comprising a body made of polyvinyl acetal material treated with a plurality of colored biocidal non-oxidating dyes to color the body a distinct color, said body section ranging from about 1 mm to about 3 mm in thickness and an outer silicone adhesive section secured to said body providing means to secure said wound dressing to a patient, said body when placed in contact with an infected wound site changing color indicating the infection; said wound dressing body having a composition by weight of about 99.5% polyvinyl acetal material, about 0.025% by weight gram negative dye and about 0.025% by weight gram positive dye.

12. A method of constructing a color change wound dressing comprising the steps of:
a). formulating a polyvinyl acetal (PVA) foam into a sheet of about 1 mm to about 3 mm thickness;
b). washing the formed PVA foam sheet to remove formaldehyde to a concentration less than 1 part per million;
c). drying out the formed PVA foam sheet to remove excess moisture;
d). applying a plurality of colored non-oxidating biocidal dyes to the PVA foam sheet, binding said dyes to the PVA at a concentration of at least 0.025 grams per gram of dye for every gram of foam;
e). cutting said PVA foam sheet to specific sized strip; and
f). securing a cover member adhesive outer section to each of said sized strip.

13. A method as claimed in claim 12 in step d). wherein said plurality of colored biocidal dyes applied to said PVA foam sheet, have at least one of said dyes being gram positive and at least one of the said being gram negative.

14. A method as claimed in claim 13 wherein said colored biocidal dyes are Methylene Blue dye and Gentian Violet.

15. A method as claimed in claim 13 wherein said colored biocidal dyes are taken from a group of dyes consisting of gram negative dyes Methylene Blue, Dimethyl Methylene Blue, New Methylene Blue, and gram positive dyes Malachite Green, Brilliant Green, Quinacrine and Acriflavine.

16. A wound dressing comprising a body section made of foamed polyurethane foam material treated with a plurality of colored non-oxidating biocidal dyes to bind the dyes to the foam material with at least one of said dyes being gram positive and at least one of the said dyes being gram negative, said dyes coloring the body a distinct color, said body ranging from about 1 mm to about 3 mm in thickness and an outer adhesive member secured to said body providing means to secure said wound dressing to a patient, said body when placed in contact with an infected wound site changing color indicating the infection.

17. A wound dressing as claimed in claim 16 wherein said wound dressing body has a composition by weight of about 99.5% polyurethane material, about 0.025% by weight Methylene Blue dye and about 0.025% by weight Gentian Violet dye.

24. (canceled)

18. A wound dressing comprising a body made of foamed polyurethane ester foam material treated with a plurality of colored non-oxidating biocidal dyes with at least one of said dyes being gram positive and at least one of the said dyes being gram negative, said dyes coloring the body section a distinct color, said body ranging from about 1 mm to about 3 mm in thickness and an outer adhesive section secured to said body providing means to secure said wound dressing to a patient, said body when placed in contact with an infected wound site changing color indicating the infection.

19. A wound dressing as claimed in claim 18 wherein said wound dressing body has a composition by weight of about 99.5% polyurethane ester material, about 0.025% by weight Methylene Blue dye and about 0.025% by weight Gentian Violet dye.

27. (canceled)

20. A wound dressing comprising a body made of foamed polyethylene material treated with a plurality of colored non-oxidating biocidal dyes with at least one of said dyes being gram positive and at least one of the said dyes being gram negative, said dyes coloring the body section a distinct color, said body ranging from about 1 mm to about 3 mm in thickness and an outer adhesive section secured to said body providing means to secure said wound dressing to a patient, said body when placed in contact with an infected wound site changing color indicating the infection.

21. A wound dressing as claimed in claim 28 wherein said wound dressing body has a composition by weight of about 99.5% polyethylene material, about 0.025% by weight Methylene Blue dye and about 0.025% by weight Gentian Violet dye.

30. (canceled)
